(19) United States
(12) Patent Application Publication
(10) Pub. No.: US 2025/0099276 A1
(43) Pub. Date: Mar. 27, 2025

(54) MAGNETIC STENT SYSTEM FOR PREVENTION OF STENT MIGRATION

(71) Applicant: THE REGENTS OF THE UNIVERSITY OF CALIFORNIA, Oakland, CA (US)

(72) Inventors: Nischal Acharya, Irvine, CA (US); Olivia Heutlinger, Irvine, CA (US); Ninh Tuan Nguyen, Orange, CA (US); Brian R. Smith, Orange, CA (US)

(21) Appl. No.: 18/896,702

(22) Filed: Sep. 25, 2024

Related U.S. Application Data
(60) Provisional application No. 63/585,130, filed on Sep. 25, 2023.

(51) Int. Cl. A61F 2/90

(52) U.S. Cl. CPC A61F 2/90; A61F 2250/0058

(57) ABSTRACT

A stent system and method to prevent stent migration. The stent system includes a magnetic stent comprising an outer surface and an inner surface, a proximal end, and a distal end with three or more magnets disposed thereon and configured to form a ring. The system may further have a magnetic ring comprising three or more magnets configured to form a ring. The magnetic ring is disposed proximally or distally to the magnetic stent, which allows magnetic repulsion to maintain the position of the stent.

### CROSS-REFERENCE TO RELATED APPLICATIONS

This application is a non-provisional and claims benefit of U.S. Provisional Application No. 63/585,130 filed Sep. 25, 2023, the specification of which is incorporated herein in their entirety by reference.

### FIELD OF THE INVENTION

The present invention features a magnetic stent system that utilizes magnetic repulsion to prevent stent migration.

### BACKGROUND OF THE INVENTION

Stents play a critical role in managing many upper gastrointestinal (GI) pathologies. Esophageal stents are the primary mode of palliation of malignant dysphagia, allowing end-of-life patients to maintain oral intake and quality of life. They are also used for similar purposes in the setting of obstruction, strictures, perforations, and fistulas. Bariatric stents are often used to treat post-bariatric surgery gastric leaks, which occur in up to 6% of patients undergoing sleeve gastrectomy and have a complication-specific mortality rate of up to 14%. When complicated by severe sepsis, this mortality rate increases to 35%. Stents have an overall success rate ranging from 80 to 95% when used for this indication. In addition to postoperative leaks, many are attempting to broaden stent usage to become a primary way of managing conditions rather than a modality to manage complications, such as the novel Full Sense device created to simulate satiety and promote weight loss without requiring an invasive surgical procedure. The stent spans across the upper surface of the inside of the stomach to stimulate the sensation of fullness, altering hormonal cues for hunger and promoting weight loss. Stents are an incredibly effective way of mitigating both surgical and pathologic complications, and their ability to be placed minimally invasively makes them an appealing target for reinventing their uses in clinical medicine. This is particularly important for the indications listed above, as patients with obesity or malignancy are poor surgical candidates and would benefit immensely from reducing operative time, length of hospital stay, and complication rates. Additionally, it reduces costs for both the hospital and the patient. The advantages of endoscopic therapies with stents are apparent; however, the complications, namely stent migration, are holding back their clinical implementation.

Complication rates for esophageal stents range from 50 to 60% in the literature, with reintervention rates nearing 50%. Specifically, migration rates are reported between 29 and 40%, with fully covered stents, plastic stents, active chemo- or radiotherapy, and benign pathology increasing the risk of migration. Migration rates for stents placed following sleeve gastrectomy average between 11 and 32%. An experiment evaluating the technical feasibility and efficacy of the Full Sense device in porcine models had promising results for weight reduction; however, 100% of the devices migrated within one week in the first trial, and 83.33% migrated during the second trial. Stent migration can lead to several subsequent complications, including re-emergence of obstructive symptoms at the site of intended placement, bleeding, perforation, fistula formation, and more. These complications often require reintervention to remove the stent or relocate it to its original position. Most of the time, this can be done endoscopically, though many cases have been reported where surgical intervention was needed due to deep migration into the bowel. In addition to causing patients emotional and financial distress, these complications and the need for reintervention increase the probability for additional adverse events to occur. This can lead to long-term effects on morbidity and mortality, especially in patients with baseline poor health status.

Many attempts have been made to combat stent migration. The use of larger diameter stents has been shown to decrease migration rates in the esophagus in some cases (rates ranging from 8-15%); however, other data has suggested that there is no difference in migration rates. Larger diameter stents were also associated with higher complication rates from hemorrhage, perforation, and fistula formation. Uncovered self-expanding metal stents (SEMS) are known to have the lowest migration rates as they generate the most friction against the GI lumen and lead to tissue hyperreactivity and ingrowth into the stent. For benign pathologies, this can make them very difficult to be removed, replaced, or repositioned. For malignant pathologies, tumor ingrowth into the stent can produce re-emergence of obstructive symptoms. Partially-covered and fully-covered SEMS protect against some of these problems, but in turn they produce higher migration rates. Other design ideas include SEMS with protuberances or wire hooks that embed into the mucosa. While this shows promise in terms of lowering migration rate, the increased friction along the esophageal mucosa increases the risk of inflammation, bleeding, and perforation.

Other approaches to preventing stent migration have focused on fixing stents at their location via clips and sutures. The Shim technique was one of the first interventions created to prevent migration, where a silk thread (or umbilical tape, dental floss, or other similar material) attached to the proximal end of the stent is advanced through the pharynx and nasal cavity and is fixed to the nostril or earlobe with tape. The most commonly used technology are endoscopic clips that are applied to the proximal or proximal and distal stent borders, fixation with an endoscopic suturing device, and the use of an over-the-scope clip system. Although these methods have improved migration rates, rates are still reported around 17-22%. More importantly, these techniques and devices are technically challenging to use and only some GI surgeons are skilled in their use. In addition, these devices have known complications of increased bleeding and perforation.

In summary, there are numerous devices of the upper GI tract that are subject to migration from their intended location due to physiological and natural factors like peristalsis, gravity, and food boluses. Migration is a significant problem that limits these devices in their clinical utility.

### BRIEF SUMMARY OF THE INVENTION

It is an objective of the present invention to provide systems, devices, and methods that prevent stent migration, as specified in the independent claims. Embodiments of the invention are given in the dependent claims. Embodiments of the present invention can be freely combined with each other if they are not mutually exclusive.

The novelty of the present invention lies in the absence of any stent on the market that utilizes repulsive magnetic forces—or magnetic forces in general—to prevent migration. Stent migration can lead to severe complications in various medical conditions, underscoring an unmet need for effective solutions. By addressing this critical gap, the implementation of repulsive magnetic forces in stent design offers significant potential for broader applications across diverse clinical scenarios.

In some embodiments, the present invention may feature a stent system, e.g., a magnetic stent system utilizing magnetic repulsion. In some embodiments, the stent system comprises a magnetic stent comprising an outer surface, a proximal end, and a distal end, and a magnetic ring comprising a plurality of magnets configured to form a ring. In some embodiments, the proximal end of the magnetic stent comprises a plurality of magnets disposed thereon and configured to form a ring. In other embodiments, the distal end of the magnetic stent comprises a plurality of magnets disposed thereon and configured to form a ring. In some embodiments, the plurality of magnetic magnets on the magnetic stent and the plurality of magnets on the magnetic ring are configured such that they repulse each other. In some embodiments, the magnetic repulsion between the plurality of magnetic magnets on the magnetic stent and the plurality of magnets on the magnetic ring prevents migration of the magnetic stent.

In some embodiments, the magnetic ring is disposed distally to the plurality of magnets on the magnetic stent such that the plurality of magnets on the magnetic stent does not extend beyond the magnetic ring.

In some embodiments, the magnetic stent comprises a medical-grade plastic, metal, or other biocompatible material. In some embodiments, the magnetic ring comprises an expandable polymer. For example, the expandable polymer may be braided around the plurality of magnets on the magnetic ring. In other embodiments, the plurality of magnets in the magnetic ring is entirely encased by a medical-grade plastic or an expandable rigid device.

Non-limiting examples of magnets that may be utilized in accordance with the present invention include but are not limited to, neodymium, samarium cobalt, alnico, ceramic, ferrite magnets, or a combination thereof.

In some embodiments, the present invention may feature a stent system, e.g., a magnetic stent system utilizing magnetic repulsion. In some embodiments, the stent system comprises a) a magnetic esophageal stent comprising an outer surface, a proximal end, and a distal end, and b) a magnetic ring comprising a plurality of magnets configured to form a ring. In some embodiments, the distal end of the magnetic esophageal stent comprises a plurality of magnets disposed thereon, wherein the plurality of magnets is configured to form a ring. In some embodiments, the magnetic esophageal stent is placed within the lumen of an esophagus, such that an outer surface of the esophageal stent engages an inner surface of the esophagus (see FIG. 3). In some embodiments, the magnetic ring is placed superior to the fundus of the stomach and inferior to the diaphragm. In some embodiments, the plurality of magnetic magnets on the magnetic stent and the plurality of magnets on the magnetic ring are configured such that they repulse each other. In some embodiments, the magnetic repulsion between the plurality of magnetic magnets on the magnetic stent and the plurality of magnets on the magnetic ring prevents migration of the magnetic stent.

In other embodiments, the present invention may feature a stent system, e.g., a magnetic stent system utilizing magnetic repulsion. In some embodiments, the stent system comprises a) a magnetic bariatric stent comprising an outer surface, a proximal end, and a distal end, and b) a magnetic ring comprising a plurality of magnets configured to form a ring. In some embodiments, the proximal end of the magnetic bariatric stent comprises a plurality of magnets disposed thereon, wherein the plurality of magnets is configured to form a ring. In some embodiments, the proximal end of the magnetic bariatric stent is positioned within the lumen of the esophagus, while the distal end is placed within the lumen of the small intestine, such as at or near the duodenum (see FIG. 4). In some embodiments, the magnetic ring is placed superior to the fundus of the stomach and inferior to the diaphragm. In some embodiments, the plurality of magnetic magnets on the magnetic stent and the plurality of magnets on the magnetic ring are configured such that they repulse each other. In some embodiments, the magnetic repulsion between the plurality of magnetic magnets on the magnetic stent and the plurality of magnets on the magnetic ring prevents migration of the magnetic stent.

Other stents may be used in accordance with the present invention, with the magnetic ring positioned at an extraluminal site, where the extraluminal location is fixed within the body.

In further embodiments, the present invention features a method of preventing stent migration. In some embodiments, the method comprises a) implanting a magnetic stent in a gastrointestinal tract and b) implanting a magnetic ring distally to the plurality of magnets on the magnetic stent. In other embodiments, the method comprises a) implanting a magnetic stent in a gastrointestinal tract and b) implanting a magnetic ring superior to the fundus of the stomach and inferior to the diaphragm. In some embodiments, the magnetic stent comprises an outer surface, a proximal end, and a distal end. In some embodiments, either the proximal end or the distal end comprises a plurality of magnets disposed thereon, wherein the plurality of magnets is configured to form a ring. In some embodiments, the magnetic ring comprises a plurality of magnets configured to form a ring. In some embodiments, the plurality of magnetic magnets on the magnetic stent and the plurality of magnets on the magnetic ring are configured such that they repulse each other and prevent migration of the magnetic stent.

In some embodiments, the magnetic stent is implanted endoscopically. In some embodiments, the magnetic ring is implanted via a laparoscopic approach, an open approach, or robotically. The magnetic stent may be implanted in the esophagus, for example, as an esophageal stent. In other embodiments, the magnetic stent may be implanted in the stomach, such as a bariatric stent.

One of the unique and inventive technical features of the present invention is the implementation of a two-part stent system comprising a magnetic ring and a stent that incorporates a plurality of magnets. Without wishing to limit the invention to any theory or mechanism, it is believed that the technical feature of the present invention advantageously prevents stent migration through the use of repulsive magnetic forces. None of the presently known prior references or works have the unique inventive technical feature of the present invention.

Moreover, the prior art teaches away from the present invention. For instance, stent migration typically occurs within the first week following initial placement, with fully covered stents and benign pathologies significantly increasing the risk. Current clinical techniques aimed at preventing migration, such as suturing and over-the-scope clip placement, are not without drawbacks. Although these methods reduce migration rates, they are both costly and require substantial time and specialized training to be implemented effectively. More importantly, the reduction in migration rates remains insufficient.

Many physicians find these techniques inefficient, cumbersome, and limited in their practical application, often reserving them for cases with a high risk of migration due to the lack of superior alternatives on the market. Additionally, further manipulation of the gastrointestinal (GI) mucosa during these procedures increases the likelihood of adverse events, such as inflammation, bleeding, and perforation.

In contrast, the inventive technical features of the present invention offer a more efficient and less invasive solution to these challenges. By utilizing a repulsive magnetic force, this device does not rely on the integrity of the esophageal tissue, which is often compromised in the patient population receiving stents. Furthermore, the additional tissue trauma of anchoring devices is avoided.

Thus, the stent system of the present invention may incorporate stents, such as upper gastrointestinal or esophageal stents, that leverage the repulsive forces of magnets to prevent migration. As stent migration is the most common complication of stent placement and is a common reason for re-intervention, the systems described herein will prevent the re-emergence of symptoms and reduce repeat procedures for stent relocation. The present design includes a stent or device that contains magnets organized in a circular fashion on the intraluminal device. The stent/device-magnetic complex will generate a magnetic field that will repel a separate magnetic field generated by a fixed magnetic ring placed under the diaphragm at the junction of the esophagus and the stomach. The interaction of the magnetic fields will produce a net force against gravity on the intraluminal device. This net upward force will allow the stent to withstand various downward forces (e.g., peristalsis, food boluses) while minimizing migration. Additionally, the present invention will help solve the problem of migration without increasing the risk of esophageal perforation, mucosal atrophy, mucosal inflammation, and fistula formation, unlike the current attempts at solving migration. The systems described herein are the first proposed solutions to leverage magnetic fields to counter downward forces contributing to intraluminal GI stent/device migration. Notably, the indications for the use of this magnetic stent/device complex with the fixed magnetic ring are incredibly broad. This technology may be used for esophageal stents in the setting of achalasia, malignant aphasia, bariatric treatment, upper GI leaks, upper GI perforation, and more. Furthermore, this technology may have additional indications in other organ systems should the fixed magnetic ring be placed at different locations.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows non-limiting embodiments of the devices of the present invention.

FIGS. 2A and 2B show non-limiting embodiments of the devices described herein.

FIG. 3 shows a non-limiting example of the stent system described herein. Specifically, FIG. 3 shows an embodiment in which the magnetic ring is disposed distally to the magnetic stent.

FIG. 4 shows a non-limiting example of the stent system described herein. FIG. 4 shows an embodiment in which the magnetic ring is disposed proximally to the magnetic stent.

FIG. 5 shows a close-up view of the magnetic ring described herein.

### DETAILED DESCRIPTION OF THE INVENTION

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. The term “comprising” means that other elements can also be present in addition to the defined elements presented. The use of “comprising” indicates inclusion rather than limitation. Stated another way, the term “comprising” means “including principally, but not necessarily solely”. Furthermore, variations of the word “comprising”, such as “comprise” and “comprises”, have correspondingly the same meanings. In one respect, the technology described herein related to the herein described compositions, methods, and respective component(s) thereof, as essential to the invention, yet open to the inclusion of unspecified elements, essential or not (“comprising”).

Although methods and materials similar or equivalent to those described herein can be used to practice or test the disclosed technology, suitable methods and materials are described below. The materials, methods, and examples are illustrative only and not intended to be limiting.

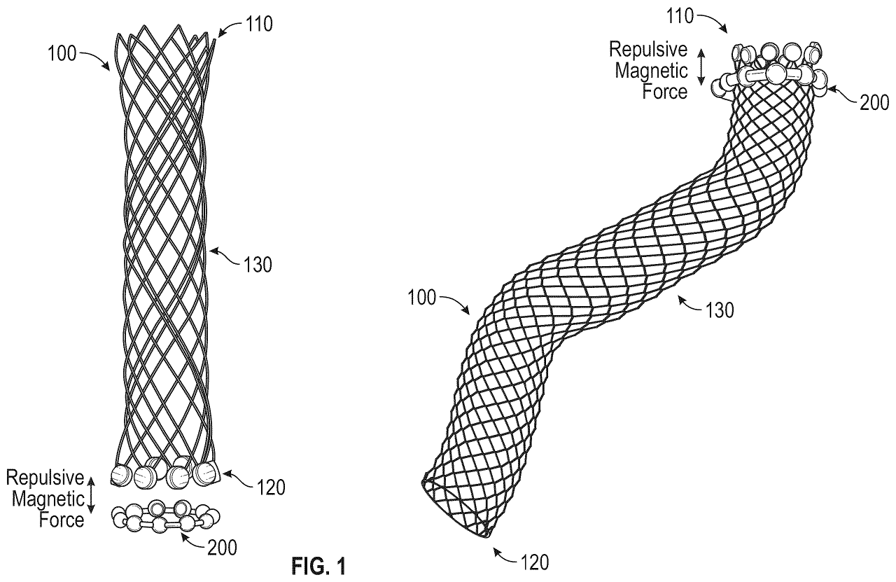

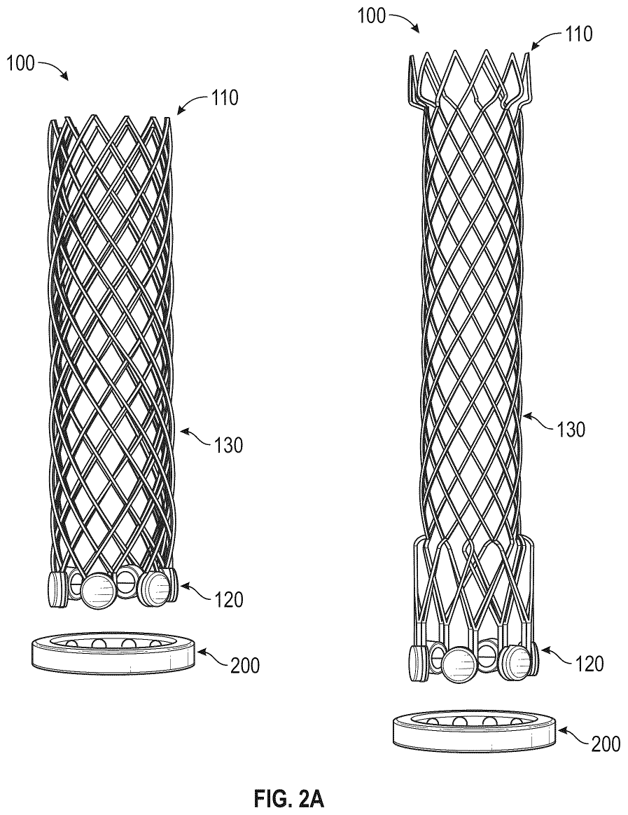

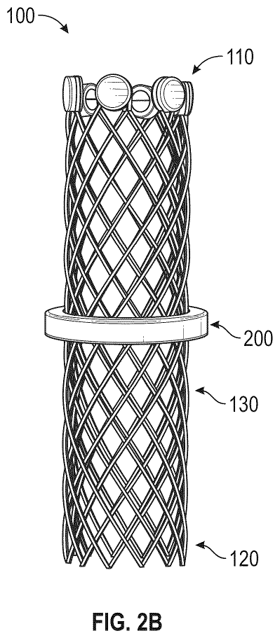

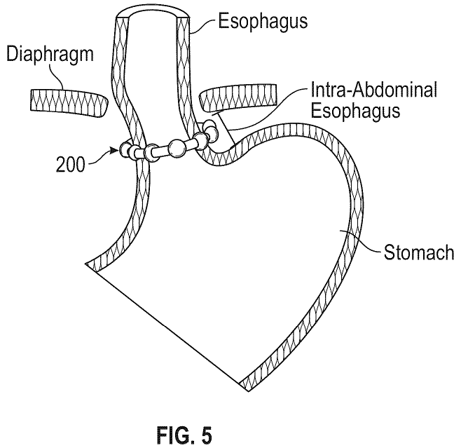

Referring now to FIGS. 1-5, the present invention features a magnetic stent system that utilizes magnetic repulsion to prevent stent migration.

### Stent System:

The stent system of the present invention comprises two parts. In some embodiments, the first component is a series of magnets (e.g., a magnetic ring (200)) arranged in a circular configuration. In a non-limiting example, the magnetic ring (200) may be designed to be positioned below the diaphragm, encircling the gastroesophageal junction. This fixed magnetic ring (200) generates a magnetic field, e.g., within the esophageal lumen. In some embodiments, the second component is a magnetic intraluminal device (100, e.g., a magnetic stent, where the magnetic intraluminal device (100) comprises a plurality of magnetic disposed at either a proximal (110) or a distal end (120)). The magnetic field produced by the magnetic intraluminal device (200) interacts with and is repelled by the magnetic field created by the fixed magnetic ring (100). The precise dimensions and magnet placement can be optimized based on the specific clinical indication tailored to the individual patient and their pathology.

The two-part stent system described herein, which utilizes magnetic repulsion, may be integrated with other stents/devices, such as stents designed to reside at the gastric fundus. In some embodiments, other devices employing this innovative technology (e.g., magnetic repulsion) will require only minor modifications to incorporate magnets oriented appropriately for optimal performance.

Non-limiting examples of stents and indications that may be utilized in conjunction with the present invention are detailed below.

For example, in patients with malignant dysphagia, an esophageal stent is typically placed; however, stent migration most commonly occurs shortly after insertion, before tumor ingrowth into the stent walls secures it in position. As advancements in cancer treatment enable these patients to live longer, their stent requirements are changing. Given their often late presentation and the presence of metastasis, there is an urgent need for solutions that effectively address migration while minimizing invasiveness, reducing procedure times, and lowering associated risks.

For patients with malignant disease who are undergoing chemo- or radiotherapy as a bridge to definitive surgical management, stents may be used to allow for oral food intake for quality of life and nutritional optimization prior to surgery. As neoadjuvant treatment begins to shrink the tumor size, the stent becomes more vulnerable to migration. In this scenario, the repulsive magnetic stent system, as described herein, that does not rely on the mucosa to hold its position will confer a better probability of reduced migration.

Alternatively, for patients with benign pathology, e.g., achalasia, benign upper gastrointestinal strictures, and obstructions, there is no aberrant tissue to invade and secure the esophageal stent in place. Typically presenting with a longer expected lifespan, these patients require stents intended for curative rather than palliative use. Consequently, they benefit more from stents designed for durability, minimizing the need for reinterventions and long-term complications.

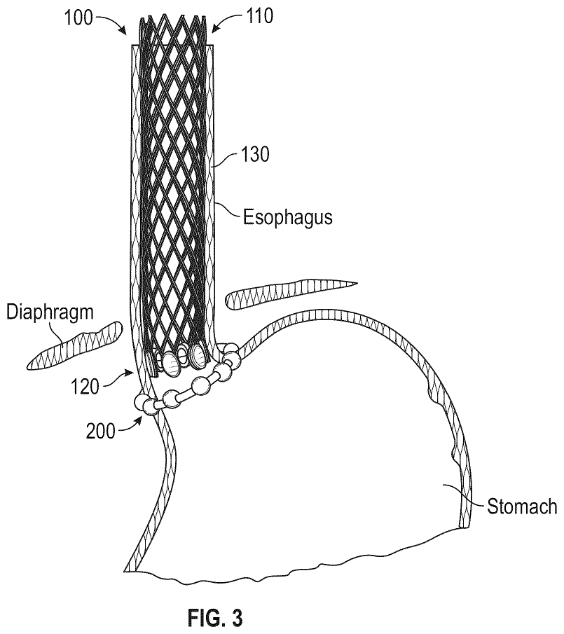

An esophageal stent designed with repulsive magnetic forces, as described herein (see FIG. 3), will help mitigate early migration and maintain stent stability throughout the course of treatment. This innovative approach ensures a minimally invasive procedure, prioritizing both patient safety and quality of life, and optimizing patient outcomes.

For patients requiring removable esophageal and bariatric stents for shorter treatment durations, the challenge lies in removing the stent after it has been in place for several weeks to months. Fully covered stents are optimal due to their surface area, which minimizes tissue embedding; however, their design also increases the risk of migration, resulting in infrequent use. The removal of a stent embedded in the esophageal or gastric wall presents technical difficulties and can lead to complications ranging from minor blood loss to intramural rupture. The migration prevention capabilities of the two-part stent system described herein will restore the clinical utility of fully covered stents, mitigating migration-related complications for patients requiring short-term stenting.

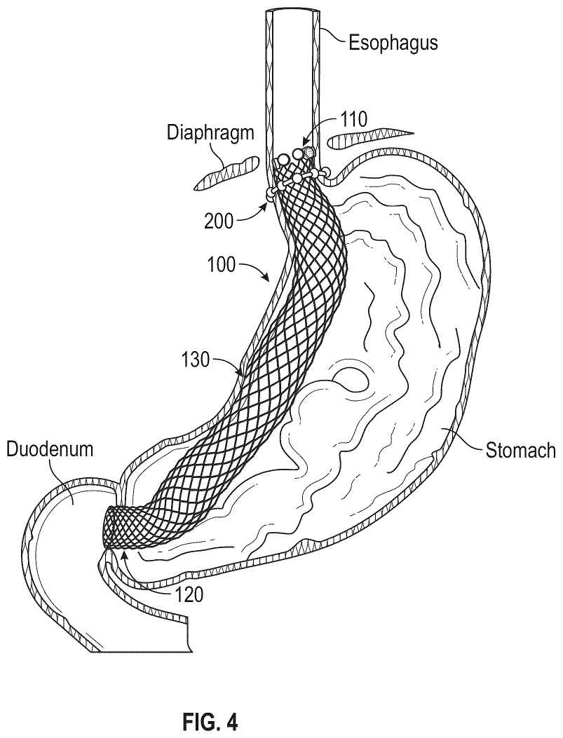

In patients pursuing surgical options for weight loss, stent migration significantly limits the effectiveness of endoscopic therapies. Implementing the systems of the present invention to prevent migration offers a viable alternative to high-risk, high-cost bariatric surgery for patients often burdened with multiple comorbidities that elevate surgical risks. Furthermore, patients undergoing surgical weight loss can have a magnetic ring placed at the time of the procedure, alleviating concerns about stent migration in the event of a gastric leak (e.g., see FIG. 4). This approach not only enhances safety but also allows for the option of utilizing endoscopic weight loss methods should they regain weight post-surgery. Operating in the same anatomical region multiple times presents technical challenges due to altered anatomy and adhesion formation, with risks exacerbated in patients with a high BMI. Given that weight regain is a common long-term outcome for 20-30% of bariatric surgery patients, this strategy could help mitigate the need for repeat surgical interventions.

By utilizing repulsive magnetic forces to hold a stent/device in place, the intraluminal portion of the device will not require any further tissue manipulation beyond the placement of the stent/device. This will eliminate the added risks of bleeding, perforation, obstructions, etc., that are consequences of both alternate strategies to prevent migration and endoscopic reintervention after stent migration. Additionally, avoiding these complications will reduce patient hospital stays and morbidity and will reduce costs for both the patient and the hospital.

Without wishing to limit the present invention to any theory or mechanism, it is believed that the present invention is unique as it incorporates a ring of magnets (magnetic ring (200)) positioned perpendicular to the long axis of the stent or device, fused with nitinol (or other suitable material) which enable repulsive forces between the magnetic ring described herein.

Additionally, without wishing to limit the present invention to any theory or mechanism, it is believed that the use of normally-poled magnets on one part of the stent system and diametrically-poled magnets on the other may generate the strongest repulsive force.

### Magnetic Stent (100)

The stent system of the present invention may feature a magnetic stent (100) comprising an outer surface (130) and an inner surface, a proximal end (110), and a distal end (120). The proximal (110) or distal end (120), depending on stent indication, comprises a plurality of magnets, e.g., three or more magnets, disposed thereon. In some embodiments, the plurality of magnets is configured to form a ring. For example, referring to FIG. 3, an esophageal stent may comprise a plurality of magnets at a distal end of the magnetic stent (100). Referring to FIG. 4 as another example, a bariatric stent may comprise a plurality of magnets at a proximal end of the magnetic stent (100).

In some embodiments, the magnetic stent (100) comprises at least one magnet disposed thereon configured to form a ring. In some embodiments, the magnetic stent (100) comprises at least three magnets disposed thereon configured to form a ring. In some embodiments, the magnetic stent (100) comprises at least five magnets disposed thereon configured to form a ring. In some embodiments, the magnetic stent (100) comprises at least ten magnets disposed thereon configured to form a ring. In some embodiments, the magnetic stent (100) comprises at least fifteen magnets disposed thereon configured to form a ring. In some embodiments, the magnetic stent (100) comprises at least twenty magnets disposed thereon configured to form a ring. The magnetic stents (100) described herein are not confined to the previously mentioned number of magnets and may incorporate any quantity of magnets suitable for effective stent placement.

In some embodiments, the plurality magnets of the magnetic stent (100) are selected from neodymium or alloys thereof, samarium cobalt or alloys thereof, alnico, ceramic or alloys thereof, or ferrite magnets or alloys thereof, or a combination thereof. In other embodiments, the plurality magnets of the magnetic stent (100) are selected from iron, nickel, cobalt, stainless steel, rare earth metals, or a combination thereof. In some embodiments, the plurality magnets of the magnetic stent (100) comprise rare earth magnets. In some embodiments, the plurality magnets of the magnetic stent (100) comprise neodymium iron. In other embodiments, the plurality magnets of the magnetic stent (100) comprise a boron magnetic core. Other magnetic materials may be used in accordance with the present invention.

In some embodiments, the plurality of magnets is normally poled (axially poled). In other embodiments, the plurality of magnets are diametrically polarized.

In some embodiments, the plurality magnets of the magnetic stent (100) are encapsulated in a coating, e.g., a biocompatible coating. In some embodiments, the plurality magnets of the magnetic stent (100) are coated. Non-limiting examples of materials that may be used to coat the plurality of magnets may include but are not limited to biocompatible materials, including but not limited to titanium, polymers, or the like.

In some embodiments, the magnetic stent (100) is a gastrointestinal stent. In other embodiments, the magnetic stent (100) is an esophageal or bariatric stent. In some embodiments, the magnetic stent (100) may be placed in the gastrointestinal (GI) tract. In some embodiments, the magnetic stent (100) may be placed in the upper GI tract. In other embodiments, the magnetic stent (100) may be placed in the lower GI tract (e.g., the small bowl, colon, or rectum). The present invention is not limited to the previously described stents and may encompass various intraluminal stent devices, including any stent placed along the gastrointestinal tract.

In some embodiments, the magnetic stent (100) comprises a medical-grade plastic, metal, or a combination thereof. In some embodiments, the magnetic stent (100) is constructed from nitinol or the like.

In some embodiments, the magnetic stent (100) comprises a diameter of about 15 to 25 mm. In other embodiments, the magnetic stent (100) comprises a diameter of about 18 to 23 mm. In some embodiments, the magnetic stent (100) comprises a length of about 50 to 200 mm. In other embodiments, the magnetic stent (100) comprises a length of about 70 to 150 mm. The present invention is not limited to the aforementioned dimensions.

In some embodiments, the magnetic stent (100) described herein is placed endoscopically.

### Magnetic Ring (200):

The stent system of the present invention may feature a magnetic ring (200) comprising a plurality of magnets, e.g., three or more magnets, configured to form a ring. Alternatively, the stent system of the present invention may feature a magnetic ring (200) comprising a singular magnet configured to form a ring.

In some embodiments, the magnetic ring (200) comprises at least one magnet. In some embodiments, the magnetic ring (200) comprises at least three magnets. In some embodiments, the magnetic ring (200) comprises at least five magnets. In some embodiments, the magnetic ring (200) comprises at least ten magnets. In some embodiments, the magnetic ring (200) comprises at least fifteen magnets. In some embodiments, the magnetic ring (200) comprises at least twenty magnets.

In some embodiments, the magnetic ring (200) comprises two or more magnets. In some embodiments, the magnetic ring (200) comprises three or more magnets. In some embodiments, the magnetic ring (200) comprises five or more magnets. In some embodiments, the magnetic ring (200) comprises ten or more magnets. In some embodiments, the magnetic ring (200) comprises twenty or more magnets. The magnetic ring (200) described herein is not restricted to the previously mentioned number of magnets and may incorporate any quantity of magnets deemed suitable for effectively preventing the migration of the magnetic stent (100).

In some embodiments, the magnetic ring (200) may be constructed using an expandable polymer. In some embodiments, the expandable polymer may be braided through numerous magnets, which will create a vertical or angled magnetic field. In other embodiments, the magnetic ring (200) may be constructed using biocompatible titanium wires or similar biocompatible metal. In certain embodiments, the expandable polymer is braided around the plurality of magnets. In other embodiments, the plurality of magnets in the magnetic ring is entirely encased by a medical-grade plastic or an expandable rigid device. In some embodiments, the magnetic ring (200) may be generated from biocompatible polymers, plastics, and/or metals to completely encase the magnets in a ring formation.

In some embodiments, the plurality of magnetics in the magnetic ring (200) is selected from neodymium or alloys thereof, samarium cobalt or alloys thereof, alnico, ceramic or alloys thereof, or ferrite magnets or alloys thereof, or a combination thereof. In other embodiments, the plurality of magnetics in the magnetic ring (200) is selected from iron, nickel, cobalt, stainless steel, rare earth metals, or a combination thereof. In some embodiments, the magnets comprise rare earth magnets. In some embodiments, the magnets comprise neodymium iron. In other embodiments, the magnetics comprise a boron magnetic core. Other magnetic materials may be used in accordance with the present invention.

In some embodiments, the plurality of magnets is normally poled (axially poled). In other embodiments, the plurality of magnets are diametrically polarized.

In some embodiments, the plurality of magnets comprising the magnetic ring (200) is encapsulated in a biocompatible coating. In some embodiments, the three or more magnets (200) are coated. Non-limiting examples of materials that may be used to coat the magnets may include but are not limited to biocompatible materials, including but not limited to titanium, polymers, or the like.

In some embodiments, the magnetic ring (200) may be inserted superior to the fundus of the stomach and inferior to the diaphragm.

In some embodiments, the magnetic ring (200) is placed via a laparoscopic or robotic approach. In other embodiments, the magnetic ring (200) is placed via an open approach.

In some embodiments, the magnetic ring (200) comprises five or more magnetics configured to form a ring. In other embodiments, the magnetic ring (200) comprises ten or more magnetics configured to form a ring. The magnets may be arranged in a circular formation around the gastroesophageal junction.

In some embodiments, the magnetic ring (200) may further comprise a locking mechanism that enables the string of magnets and polymer, plastic, titanium, or other biocompatible material to securely encircle the esophagus between the diaphragm and the fundus of the stomach without compressing the esophagus and allowing for a food bolus to enter the stomach. For example, the magnetic ring (200) may include an opening or break that allows it to be positioned around the esophagus, with a locking mechanism to securely re-form the ring shape.

In other embodiments, a series of three or more magnets may be encased in polymer, titanium, or other biocompatible material such that the ring is formed with the magnets embedded within the rigid structure. The magnetic field generated by an individual magnet will be perpendicular to the length of the magnet. The magnets will be angled and oriented such that the resulting magnetic field generated by the circle of magnets will be superior to the location of the ring.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

Reference numbers recited herein, in the drawings, and in the claims are solely for ease of examination of this patent application and are exemplary. The reference numbers are not intended in any way to limit the scope of the claims to the particular features having the corresponding reference numbers in the drawings.

What is claimed is:

1. A stent system comprising:
a) a magnetic stent (100) comprising an outer surface (130), a proximal end (110), and a distal end (120); wherein either the proximal end (110) or the distal end (120) comprises a plurality of magnets disposed thereon; wherein the plurality of magnets are configured to form a ring; and
b) a magnetic ring (200) comprising a plurality of magnets configured to form a ring;
wherein the plurality of magnetic magnets on the magnetic stent (100) and the plurality of magnets on the magnetic ring (200) are configured such that they repulse each other.

2. The stent system of claim 1, wherein the magnetic ring (200) is disposed distally to the plurality of magnets on the magnetic stent (100).

3. The stent system of claim 1, wherein the plurality of magnets on the magnetic stent (100) does not extend beyond the magnetic ring (200).

4. The stent system of claim 1, wherein the magnetic stent (100) comprises a medical-grade plastic, metal, or other biocompatible material.

5. The stent system of claim 1, wherein the magnetic ring (200) comprises an expandable polymer.

6. The stent system of claim 5, wherein the expandable polymer is braided around the plurality of magnets on the magnetic ring (200).

7. The stent system of claim 1, wherein the plurality of magnets in the magnetic ring (200) is entirely encased by a medical-grade plastic, or wherein the plurality of magnets in the magnetic ring (200) is entirely encased with an expandable rigid device.

8. The stent system of claim 1, wherein the plurality of magnets are selected from neodymium, samarium cobalt, alnico, ceramic, or ferrite magnets, or a combination thereof.

9. The stent system of claim 1, wherein the magnetic stent (100) comprises a bariatric stent.

10. The stent system of claim 1, wherein the magnetic stent (100) comprises an esophageal stent.

11. The stent system of claim 10, wherein the esophageal stent is placed within the lumen of an esophagus, such that an outer surface of the esophageal stent engages an inner surface of the esophagus.

12. The stent system of claim 11, wherein the magnetic ring (200) is placed superior to a fundus of a stomach and inferior to a diaphragm.

13. The stent system of claim 1, wherein the magnetic ring (200) is placed at an extraluminal location, wherein the extraluminal location is at a fixed location within the body.

14. The stent system of claim 1, wherein the magnetic repulsion between the plurality of magnetic magnets on the magnetic stent (100) and the plurality of magnets on the magnetic ring (200) prevents migration of the magnetic stent.

15. A method of preventing stent migration comprising:
a) implanting a magnetic stent (100) in a gastrointestinal tract, wherein the magnetic stent (100) comprises an outer surface (130), a proximal end (110), and a distal end (120); wherein either the proximal end (110) or the distal end (120) comprises a plurality of magnets disposed thereon; wherein the plurality of magnets are configured to form a ring; and
b) implanting a magnetic ring (200) distally to the plurality of magnets on the magnetic stent (100), wherein the magnetic ring (200) comprises a plurality of magnets configured to form a ring;
wherein the plurality of magnetic magnets on the magnetic stent (100) and the plurality of magnets on the magnetic ring (200) are configured such that they repulse each other and prevent migration of the magnetic stent.

16. The method of claim 15, wherein the magnetic stent (100) is implanted endoscopically.

17. The method of claim 15, wherein the magnetic ring (200) is implanted via a laparoscopic approach or open approach, or robotically.

18. The method of claim 15, wherein the magnetic stent (100) is implanted in an esophagus.

19. The method of claim 15, wherein the magnetic stent (100) is implanted in a stomach.
